(19) United States
(12) Patent Application Publication
(10) Pub. No.: US 2023/0151438 A1
(43) Pub. Date: May 18, 2023

(54) VIRAL DETECTION ASSAY

(71) Applicant: The Council of the Queensland Institute of Medical Research, Herston (AU)

(72) Inventors: Rajiv Khanna, Herston (AU); Jacqueline Burrows, Albany Creek (AU)

(21) Appl. No.: 17/251,393

(22) PCT Filed: Jun. 12, 2019
(86) PCT No.: PCT/IB2019/000811

§ 371 Date: Dec. 11, 2020

Related U.S. Application Data
(60) Provisional application No. 62/684,277, filed on Jun. 13, 2018.

(51) Int. Cl. C12Q 1/70; C12N 5/0783; C12N 15/86

(52) U.S. Cl. CPC C12Q 1/701; C12N 5/0638; C12N 15/86; C12N 2710/10343; C12N 2710/16022; C12N 2502/11; C12N 2502/1121

(57) ABSTRACT

Provided herein are methods for preparing and characterizing CTL cultures and preparations. Also provided herein are methods of determining the presence or absence of trace amounts of viral vectors following the preparation of CTLs.

### RELATED APPLICATIONS

This application is the 371 National Stage of International Patent Application No. PCT/IB2019/000811, filed Jun. 12, 2019; which claims the benefit of priority to U.S. Provisional Patent Application Ser. No. 62/684,277 filed Jun. 13, 2018, which is incorporated by reference in its entirety.

### SEQUENCE LISTING

The application contains a Sequence Listing which has been submitted electronically in ASCII format and is hereby incorporated by reference in its entirety. Said ASCII copy, created on Dec. 1, 2020, is named OAH-01825_SL.txt and is 14,221 bytes in size.

### BACKGROUND

Adoptive immunotherapy involves implanting or infusing disease-specific T cells, such as cytotoxic T cells (CTLs), into individuals with the aim of recognizing, targeting, and destroying disease-associated cells. Adoptive immunotherapies (e.g., T-cell therapies) have become a promising approach for the treatment of many diseases and disorders, including cancer, post-transplant lymphoproliferative disorders, infectious diseases (e.g., viral infections), and autoimmune diseases.

Epstein Barr Virus (EBV), also known as human herpesvirus 4, is a ubiquitous herpes virus. Recently, it has been shown that exposure to EBV can predispose or otherwise play a role in the pathogenesis of autoimmune diseases, including multiple sclerosis (MS) and systemic autoimmune disease (SAD), and inflammatory bowel disease (IBD). Such pathologies (i.e., MS, SAD and IBD), arise from abnormal immune response against the body's own tissue. MS is characterized by the degradation of the myelin, a protective lipid shell surrounding nerve fibers, by the body's own immune cells. SADs are a group of connective tissue diseases with diverse symptoms that include rheumatoid arthritis (RA), systemic lupus erythematosus (SLE) and Sjögren's syndrome (SS). IBDs are a group of inflammatory conditions of the colon and small intestine that include Crohn's disease, celiac disease, and ulcerative colitis. For example, recent studies have shown that individuals diagnosed with MS show higher levels of EBV related proteins in B cells aggregated in nerve tissue than healthy individuals.

Immuno-surveillance by CTLs also plays a critical role in the detection and killing of a wide range of malignant cells (Gottschalk et al. 2005. Leuk Lymphoma. 46:1-10; Ochsenbein et al. 2002. Cancer Gene Ther. 9:1043-10). Survival and spread of malignant cells has been associated with the ability of such cells to evade recognition by CTLs (Bubenik et al. 2003. Oncol. Rep. 10:2005-2008; Rees et al. 1999. Cancer Immunol. Immunother. 48:374-381). In particular, antigen processing and presentation functions remain intact in the EBV-associated malignancies such as Hodgkin's lymphoma and nasopharyngeal carcinoma (Khanna et al. 1998. Cancer Res. 58: 310-314, Lee et al. 1998. Blood 92: 1020-1030). Recent studies have suggested that immuno-surveillance (and subsequent response) may be subverted by regulatory T cell-mediated suppression of the EBV-specific T cells, leading to failure to clear malignant cells. Therefore, augmentation of T cell responses against cancer-associated antigens, via the adoptive transfer of CTLs may offer attractive alternatives to current treatment strategies.

The association between viral infection (e.g., by EBV) and the pathogenesis of multiple conditions, e.g., cancers and autoimmune diseases, provided the impetus for several groups to develop the production of allogeneic (see, e.g., WO 2017/203368, hereby incorporated by reference) and autologous (see, e.g., Pender et al., Multiple Sclerosis Journal. 2014; 20(11): 1541-1544) antigen-specific CTLs. The development of improved methods for ensuring that such CTL preparations are of suitable quality for use in human medicine (e.g., substantially free of residual viral vector, such as adenovirus) prior to administration are of vital importance.

### SUMMARY OF THE INVENTION

Provided herein are methods of generating allogeneic or autologous cytotoxic T cells (CTLs) that express a T cell receptor that specifically binds to an EBV peptide presented on a class I MHC. In some embodiments, the CTLs are generated by incubating a sample comprising CTLs (responder cells, e.g., a PBMC sample) with antigen presenting cells (APCs, i.e., stimulator cells) presenting an EBV peptide on a class I MHC (e.g., a class I MHC encoded by an HLA allele that is present in the subject), thereby inducing proliferation of peptide-specific CTLs in the sample. In some embodiments, the stimulator cells are made to present the EBV peptide by incubating PBMCs with a viral vector encoding for the EBV peptide, thereby inducing the stimulator cells to present the EBV peptide. In some embodiments, the EBV peptide comprises a LMP1 peptide or a fragment thereof, a LMP2A peptide or fragment thereof, and/or an EBNA1 peptide or fragment thereof. In some embodiments, the EBV peptide comprises a sequence listed in Table 1. In some embodiments, the viral vector is a recombinant replication incompetent adenovirus (e.g., AdE1-LMPpoly). In some embodiments, the stimulator cells may be B cells, antigen-presenting T cells, dendritic cells, or artificial antigen-presenting cells (e.g., a cell line expressing CD80, CD83, 41BB-L and/or CD86, such as aK562 cells). In some embodiments, the stimulator cells are irradiated.

Also provided herein are methods of determining the presence or absence of trace amounts of the viral vector (e.g., AdE1-LMPpoly) following preparation of CTLs expressing a T cell receptor that specifically binds to an EBV peptide presented on a class I MHC. Alongside an assay detecting the presence of replication competent adenovirus or AdE1-LMPpoly (viral replication assay), a highly sensitive reverse transcriptase polymerase chain reaction (RT-PCR) technique is used to test for the presence of viral mRNA transcripts indicative of active virus. The active virus might be present at early stages of the manufacturing process, as this leads to presentation of viral epitopes to T cells by antigen-presenting cells. However, this method can identify the presence or absence of viral mRNA at later time points.

In some aspects, provided herein are methods for assessing whether a preparation of responder cells comprising cytotoxic T lymphocytes (CTLs) is essentially free of an active virus. In some embodiments, a culture of responder cells is prepared comprising virally transduced stimulator cells. In some such embodiments, at least one sample is collected from said culture and assessed for the presence of active virus. The ability of said sample to form viral plaques on a plurality of reporter cell lines is assessed, e.g., in a viral replication assay. Preferably, the presence or absence of a viral mRNA in said sample is determined. In some such embodiments, if the samples do not form viral plaques and/or said viral mRNA is not detected, the culture is identified as being essentially free of the active virus. In some aspects, provided herein are methods for generating a preparation comprising cytotoxic T lymphocytes (CTLs) essentially free of an active virus. In certain embodiments, antigen-specific CTL proliferation is induced by culturing responder cells with virally transduced stimulator cells for a predetermined incubation time. Preferably, stimulator cells are transduced with one or more viral vectors encoding one or more antigens. In some embodiments, a sample of the culture is collected and assessed for the ability to form viral plaques on a plurality of reporter cell lines. Preferably, a sample of the culture is assessed for the presence or absence of a viral mRNA. The preparation is identified as being essentially free of an active virus if the sample does not form viral plaques and/or the presence of the viral mRNA is not detected. In preferred embodiments, the preparation is reseeded, excluded or discarded according to a predetermined protocol if the preparation is identified as not being free of the active virus.

In certain aspects, provided herein are methods for identifying a therapeutic preparation of cytotoxic T lymphocytes (CTLs) as suitable for administration. In some embodiments, a sample of a therapeutic preparation of CTLs is obtained and assessed for the ability to form viral plaques on a plurality of reporter cell lines. Preferably, the presence or absence of a viral mRNA in the sample is determined. The preparation is identified as suitable for administration to the recipient if the sample does not form viral plaques and/or viral mRNA is not detected.

### BRIEF DESCRIPTION OF THE DRAWINGS

FIG. 1 shows a flow diagram outlining procedure and experimental groups.

FIG. 2 shows detection of AdE1-LMPpoly E1PT mRNA transcripts by RT-PCR over time course, post infection. Data was obtained for three donors. ND indicates no data (insufficient RNA extracted).

FIG. 3 Detection of housekeeping gene transcripts for loading control. Ct indicates cycle threshold (i.e. PCR cycle that detection of gene becomes possible). ND indicates no data collected (insufficient RNA extracted).

### DETAILED DESCRIPTION

### General

Manufacture of CTLs expressing a T cell receptor that specifically binds to a peptide presented on a class I MHC requires T cell expansion against defined antigens. Antigen delivery may be via viral transduction of a sample of peripheral blood mononuclear cells (PBMCs) from healthy donors with recombinant virus. The transduced PBMCs act as antigen-presenting cells and are referred to as “stimulators”. The viral vector used to transduce stimulators may be a recombinant, replication incompetent virus (e.g., an adenovirus such as AdE1-LMPpoly). A separate sample of PBMCs (e.g., PBMCs from the same donor that are not used for transduction, or a sample of PBMCs from a different healthy donor) are termed “responders” and contain T cells that become the active component of CTLs expressing a T cell receptor that specifically binds to a peptide presented on a class I MHC. The antigen-presenting cells within the stimulator fraction will present the antigen to T cells, thus activating and inducing proliferation of the antigen-specific T cells of the Responder fraction.

Accordingly, provided herein are methods of generating allogeneic or autologous cytotoxic T cells (CTLs) that express a T cell receptor that specifically binds to, for example, an EBV peptide presented on a class I MHC. In some embodiments, APCs are generated via viral infection of PBMC (stimulator cells), e.g., by an adenoviral vector, such as AdE1-LMPpoly. The AdE1-LMPpoly vector encodes a polyepitope of defined CTL epitopes from LMP1 and LMP2 fused to a Gly-Ala repeat-depleted EBNA1 sequence. The AdE1-LMPpoly vector is described, for example, in Smith et al., Cancer Research 72:1116 (2012); Duraiswamy et al., Cancer Research 64:1483-9 (2004); and Smith et al., J. Immunol 117:4897-4906 (2006), each of which is hereby incorporated by reference. In some embodiments, the stimulator cells are mixed with non-infected PBMC (responders) containing T cells to present the EBV polyepitopes to the T cells. In some embodiments, virus-specific T cells presented with EBV polyepitopes are activated and induced for proliferation.

In certain aspects of the invention, provided herein are methods for identifying a preparation of responder cells comprising cytotoxic T lymphocytes (CTLs) as essentially free of an active virus. In some embodiments, a culture of responder cells comprising virally transduced stimulator cells is prepared. In certain embodiments, at least one sample is collected from said culture and assessed for the presence of active virus. Preferably, the ability of said sample to form viral plaques on a plurality of reporter cell lines is assessed, e.g., in a viral replication assay. More preferably, the presence or absence of a viral mRNA in said sample is determined. In some such embodiments, if the samples do not form viral plaques and/or said viral mRNA is not detected, the culture is identified as being essentially free of the active virus. In certain aspects, provided herein are methods for generating a preparation comprising cytotoxic T lymphocytes (CTLs) essentially free of an active virus. In certain embodiments, antigen-specific CTL proliferation is induced by culturing responder cells with virally transduced stimulator cells for a predetermined incubation time. Preferably, stimulator cells are transduced with one or more viral vectors encoding one or more antigens. In some embodiments, a sample of the culture is collected and assessed for the ability to form viral plaques on a plurality of reporter cell lines. Preferably, a sample of the culture is assessed for the presence or absence of a viral mRNA. The preparation is identified as being essentially free of an active virus if the sample does not form viral plaques and/or the presence of the viral mRNA is not detected. Preferably, the preparation is reseeded, excluded or discarded according to a predetermined protocol if the preparation is identified as not being free of the active virus.

In certain aspects, provided herein are methods for identifying a therapeutic preparation of cytotoxic T lymphocytes (CTLs) as suitable for administering to a recipient. In some embodiments, a sample of a therapeutic preparation of CTLs is obtained and assessed for the ability to form viral plaques on a plurality of reporter cell lines. Preferably, the presence or absence of a viral mRNA in the sample is determined. In such embodiments, the preparation is identified as suitable for administering to the recipient if the sample does not form viral plaques and/or said viral mRNA is not detected. Preferably, the viral plaques and viral mRNA are due to an adenovirus. Most preferably, such methods as provided herein are performed prior to administering the CTLs to a recipient.

In some embodiments, the responder cells and the stimulator cells are each derived from peripheral blood mononuclear cells (PBMC). In some such embodiments, the responder cells and the stimulator cells are each derived from PBMCs from the same donor. In other embodiments, the responder cells and the stimulator cells are each derived from PBMCs from different donors.

Prior to presentation to responder cells, stimulator cells are transduced with a viral vector, preferably an adenoviral vector comprising a nucleic acid sequence encoding a herpesvirus antigen. In some such embodiments, the adenoviral vector is replication incompetent. More preferably, the adenoviral vector comprises a nucleic acid sequence encoding one or more EBV antigens. The one or more EBV antigens may comprise an LMP1 peptide or fragment thereof, an LMP2A peptide or fragment thereof, and/or an EBNA1 peptide or fragment thereof. Most preferably, the adenoviral vector is AdE1-LMPpoly and encodes a polyepitope of defined CTL epitopes from LMP1 and LMP2 fused to a Gly-Ala repeat-depleted EBNA1 sequence. In some embodiments, the stimulator cells are incubated with one or more cytokines prior to culturing with (i.e., presentation to) responder cells (e.g., non-infected PBMC). Such stimulator cells may comprise B cells, antigen-presenting T-cells, dendritic cells, artificial antigen-presenting cells, and/or aK562 cells.

Though antigen-specific T cells achieve activation and proliferation when presented with antigen by the stimulator fraction, such stimulator cells are not desirable in the final harvested CTL product. Moreover, in order to minimize the risk of any viral recombination events in proliferating cells leading to formation of competent virus, the stimulator PBMC are treated and/or modified prior to culturing with responders so as to inhibit their proliferation, e.g., by irradiation with gamma rays or exposure to an agent such as mitomycin C. In such culture conditions, virus specific T-cells of the responder cells are presented with EBV polyepitopes by non-proliferating stimulator cells. In some such embodiments, the culture is maintained for at least 24 hours, at least 5 days, at least 8 days, at least 11 days, at least 14 days, at least 17 days, or at least 20 days prior to collecting samples. Preferably, the culture is essentially free from active virus following an incubation time of 8 days. In certain embodiments, the culture is re-seeded. In preferred embodiments, the culture is reseeded with antigenic re-stimulation (e.g. with fresh, non-proliferating, antigen-presenting stimulator cells). In some such embodiments, the culture is re-seeded as necessary and said re-seeded culture is maintained for at least 24 hours, at least 5 days, at least 8 days, at least 11 days, at least 14 days, at least 17 days, or at least 20 days prior to collecting samples. One of skill in the art would appreciate that cell proliferation in culture may vary and is limited by requirements for nutrients and oxygen, and by accumulation of waste products such as carbon dioxide and lactic acid. As such, one of skill in the art would be able to empirically determine appropriate culture and (if necessary) re-seeding schedules, to achieve the CTLs of the invention. In certain embodiments, the culture is maintained until a predetermined CTL harvesting date. In some such embodiments, assessment of the culture and determination of the presence or absence of an active virus occurs prior to and/or on the day of harvest. Most preferably, the cultures and/or preparations disclosed herein are essentially free from active virus at the time of CTL harvest.

In some embodiments, assessment of responder (CTL) cultures and detection of active virus is performed by a viral replication assay. In certain embodiments, a sample of the responder culture is incubated with a reporter cell line. In some such embodiments, the sample comprises the culture supernatant (e.g. media collected from the responder culture). Preferably, the sample is prepared from cell lysate. In certain preferred embodiments, the reporter cell line is a permissive cell line that allows the growth of a replication-incompetent virus. For example, a reporter cell line that expresses the genes encoded by the E1 region of adenovirus (e.g. AD293) complements the E1 deletion in recombinant adenoviral vectors (e.g., AdE1-LMP poly). In certain embodiments, the reporter cell line is a non-permissive cell line that is susceptible to a replication competent virus, such as A549 cells which do not produce endogenous E1 and thus cannot support replication of recombinant adenoviral vectors comprising an E1 deletion. Accordingly, following incubation with the responder culture samples, reporter cell lines are assessed for cytopathic effects by microscopic inspection. Cytopathic effects include observations such as rounding of cells and/or clearing of the monolayer. The cytopathic effect may present as a viral plaque (e.g., a localized region of cell destruction) in the monolayer of the reporter cell culture. Accordingly, in preferred embodiments, the preparation is identified as essentially free of active virus by confirming that the sample does not generate cytopathic effects or form viral plaques in a reporter cell line susceptible to the virus. One of skill in the art would appreciate that an appropriate viral vector and reporter cell line would be chosen for inclusion in the practice of the invention based on the appearance and quality of such cytopathic effects. For example, the appearance of viral plaques depends on the reporter cell line, virus, and the culturing conditions. Highly virulent or lytic virus give clear plaques while virus that only kills a fraction of the reporter line, or only reduces the rate of cell growth, gives turbid plaques.

In certain embodiments, a nucleic acid amplification technique is used to demonstrate that trace amounts of active virus are not present in the CTLs. In some embodiments, the technique used to detect trace amounts of the active virus is reverse transcriptase polymerase chain reaction (RT-PCR). In certain preferred embodiments, quantitative RT-PCR is used to demonstrate that trace amounts of active adenovirus are eventually absent from CTL cultures or from the final CTL product. RT-PCR is a technique by which RNA molecules (or targeted sequences thereof) are converted into their complementary DNA (cDNA) sequences by any one of several reverse transcriptases in conjunction with appropriately designed primers, wherein the newly synthesized cDNA is amplified using PCR procedures. A transcript must be present in a sample (e.g., culture supernatant or cell lysate) to support reverse transcription and amplification by PCR. In some embodiments the RNA is extracted from the sample for use in RT-PCR. Given the extreme sensitivity of PCR, it is particularly suited to detection and quantification of transcripts present in extremely low abundance. In some embodiments, the quantification of mRNA using RT-PCR is achieved as a one-step reaction wherein the entire reaction from reverse transcriptase reaction/cDNA synthesis to PCR amplification occurs in a single vessel. In other embodiments, the quantification of mRNA using RT-PCR is achieved as a two-step reaction wherein the reverse transcriptase reaction and PCR amplification are performed sequentially, in separate vessels. In preferred embodiments, the technique used is quantitative RT-PCR, wherein amplification reactions are characterized by the point in time during PCR cycling when amplification of a target sequence is first detected rather than the amount of target sequence accumulated after a fixed number of cycles. More preferably, the amplification product is detected using fluorescent dyes. For example, an oligonucleotide probe may be constructed comprising a reporter fluorophore on the 5′ end and a quencher dye on the 3′ end. While the probe is intact, the proximity of the quencher dye greatly reduces the fluorescence emitted by the reporter dye by fluorescence resonance energy transfer (FRET) through space. In preferred embodiments, the oligo probe comprises SEQ ID NO: 29. If the target sequence (e.g., the junction of EBNA1 and the E1-LMPpoly polyepitope of AdE1-LMPpoly) is present, the probe anneals downstream from one of the primer sites and is cleaved by the 5′ nuclease activity of Taq DNA polymerase as the primer is extended. The cleavage of the probe separates the fluorescent dye from the quencher dye, thus increasing the quantifiable fluorescent signal intensity with each amplification cycle of the target sequence. In some embodiments, the primers are designed to hybridize to and amplify a nucleic acid sequence specific to AdE1-LMPpoly or a nucleic acid sequence common to wild-type endogenous virus. Preferably, the primer set is designed to amplify a nucleic acid sequence of AdE1-LMPpoly at the junction of EBNA1 and the E1-LMPpoly polyepitope. In more preferable embodiments, the primers comprise SEQ ID NO: 27 and SEQ ID NO: 28. Given a known target sequence to be amplified, the choice and design of primers to practice the invention as described herein are within the purview of the skilled artisan.

In preferred embodiments, the CTLs are harvested from culture for processing prior to use and/or storage if culture samples do not form viral plaques on an appropriate reporter cell line and/or the sample does not have detectable viral mRNA. Conversely, in some embodiments, the culture is reseeded, excluded and/or discarded according to a predetermined protocol if culture samples do form viral plaques on an appropriate reporter cell line and/or viral mRNA is detected in said samples.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “binding” or “interacting” refers to an association, which may be a stable association, between two molecules, e.g., between a TCR and a peptide/MHC, due to, for example, electrostatic, hydrophobic, ionic and/or hydrogen-bond interactions under physiological conditions.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid; or cells from any time in gestation or development of the subject.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epidermal keratinocytes and B-lymphocytes. Lymphokines are generally referred to as being produced by lymphocyte cells. Examples of cytokines include, but are not limited to, Interleukin-1 (IL-1), Interleukin-2 (IL-2), Interleukin-6 (IL-6), Interleukin-8 (IL-8), Tumor Necrosis Factor-alpha (TNFα), and Tumor Necrosis Factor beta (TNFβ).

The term “epitope” means a protein determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of a TCR to bind to a peptide presented on an MHC (e.g., class I MHC or class II MHC). Typically, a TCR specifically binds to its peptide/MHC with an affinity of at least a K_{D} of about 10⁻⁴ M or less, and binds to the predetermined antigen/binding partner with an affinity (as expressed by K_{D}) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Peptides

In certain aspects, provided herein are methods of generating allogeneic or autologous CTLs expressing TCRs that specifically bind to peptides comprising EBV epitopes presented on class I MHC for treating autoimmune disorders (e.g., MS, SAD and/or IBD). In some embodiments, CTLs are generated by incubating a sample comprising CTLs (i.e., a PBMC sample) with antigen-presenting cells (APCs) that present one or more of the EBV epitopes described herein (e.g., APCs that present a peptide described herein comprising a EBV epitope on a class I MHC complex).

In some embodiments, the peptides provided herein comprise a sequence of any EBV viral protein (e.g., a sequence of at least 5, 6, 7, 8, 9, 10, 11, 12, 13, 14, 15, 16, 17, 18, 19 or 20 contiguous amino acids of any EBV protein). In some embodiments, the peptides provided herein comprise no more than 25, 20, 19, 18, 17, 16, 15, 14, 13, 12, 11 or 10 contiguous amino acids of the EBV viral protein.

In some embodiments, the peptides provided herein comprise a sequence of LMP1 (e.g., a sequence of at least 5, 6, 7, 8, 9, 10, 11, 12, 13, 14, 15, 16, 17, 18, 19 or 20 contiguous amino acids of LMP1). In some embodiments, the peptides provided herein comprise no more than 25, 20, 19, 18, 17, 16, 15, 14, 13, 12, 11 or 10 contiguous amino acids of LMP1. An exemplary LMP1 amino acid sequence is provided below (SEQ ID NO: 1):

| | |
|---|---|
| 1 | mdldlergpp gprrpprgpp Issyialall |
| | llllallfwl yiimsnwtgg allvlyafal |
| 61 | mlviiiliif ifrrdllcpl galcllllmi |
| | tlllialwnl hgqalylgiv ififgcllvl |
| 121 | giwvyfleil wrlgatiwql lafflaffld |
| | illliialyl qqnwwtilvd llwlllflai |
| 181 | liwmyyhgqr hsdehhhdds iphpqqatdd |
| | ssnhsdsnsn egrhhllvsg agdapplcsq |
| 241 | nlgapgggpd ngpqdpdntd dngpqdpdnt |
| | ddngphdplp qdpdntddng pqdpdntddn |
| 301 | gphdpiphnp sdsagndggp pnlteevenk |
| | ggdrgppsmt dggggdphlp tlllgtsgsg |
| 361 | gddddphgpv qlsyyd |

In some embodiments, the peptides provided herein comprise a sequence of LMP2A (e.g., a sequence of at least 5, 6, 7, 8, 9, 10, 11, 12, 13, 14, 15, 16, 17, 18, 19 or 20 contiguous amino acids of LMP2A). In some embodiments, the peptides provided herein comprise no more than 25, 20, 19, 18, 17, 16, 15, 14, 13, 12, 11 or 10 contiguous amino acids of LMP2A. An exemplary LMP2A amino acid sequence is provided below (SEQ ID NO: 2):

| | |
|---|---|
| 1 | mgslemvpmg agppspggdp dgddggnnsq |
| | ypsasgsdgn tptppndeer esneeppppy |
| 61 | edldwgngdr hsdyqplgnq dpslylglqh |
| | dgndglpppp ysprddssqh iyeeagrgsm |
| 121 | npvclpviva pylfwlaaia ascftasvst |
| | vvtatglals llllaavass yaaaqrkllt |
| 181 | pvtvltavvt ffaicltwri edppfnsllf |
| | allaaagglq giyvlvmlvl lilayrrrwr |
| 241 | rltvcggimf lacvivlivd avlqlspllg |
| | avtvvsmtll llafvlwlss pgglgtlgaa |
| 301 | lltlaaalal laslilgtin ittmfllmll |
| | wtlvvllics scsscpltki llarlflyal |
| 361 | allllasali aggsilqtnf kslsstefip |
| | nlfcmllliv agilfilail tewgsgnrty |
| 421 | gpvfmclggl Itmvagavwl tvmtntllsa |
| | wiltagflif ligfalfgvi rccryccyyc |
| 481 | itleseerpp tpyrntv |

In some embodiments, the peptides provided herein comprise a sequence of EBNA1 (e.g., a sequence of at least 5, 6, 7, 8, 9, 10, 11, 12, 13, 14, 15, 16, 17, 18, 19 or 20 contiguous amino acids of EBNA1). In some embodiments, the peptides provided herein comprise no more than 25, 20, 19, 18, 17, 16, 15, 14, 13, 12, 11 or 10 contiguous amino acids of EBNA1. An exemplary EBNA1 amino acid sequence is provided below (SEQ ID NO: 3):

| | |
|---|---|
| 1 | pffhpvgead yfeylqeggp dgepdvppga |
| | ieqgpaddpg egpstgprgq gdggrrkkgg |
| 61 | wfgkhrgqgg snpkfeniae glrvllarsh |
| | vertteegtw vagvfvyggs ktslynlrrg |
| 121 | talaipqcrl tplsrlpfgm apgpgpqpgp |
| | Iresivcyfm vfiqthifae vlkdaikdlv |
| 181 | mtkpaptcni kvtvcsfddg vdlppwfppm |
| | vegaaaegdd gddgdeggdg degeegqe |

In some embodiments, the peptide comprises the sequence of an epitope listed in Table 1.

TABLE 1
Exemplary EBV viral protein epitopes

| Epitope Sequence | HLA Restriction | SEQ ID NO. |
|---|---|---|
| CLGGLLTMV | A*02 | 4 |
| FLYALALLL | A*02 | 5 |
| YLQQNWWTL | A*02, A*68, A*69 | 6 |
| YLLEMLWRL | A*02 | 7 |
| ALLVLYSFA | A*02 | 8 |
| LLSAWILTA | A*0203 | 9 |
| LTAGFLIFL | A*0206 | 10 |
| SSCSSCPLSKI | A*ll | 11 |
| PYLFWLAA | A*23, A*24, A*30 | 12 |
| TYGPVFMCL | A*24 | 13 |
| VMSNTLLSAW | A*25 | 14 |
| CPLSKILL | B*08 | 15 |
| RRRWRRLTV | B*27 | 16 |
| IEDPPFNSL | B*40 | 17 |
| IALYLQQNW | B*57, B*58 | 18 |
| MSNTLLSAW | B*58 | 19 |
| VLKDAIKDL | A*0203 | 20 |
| RPQKRPSCI | B*07 | 21 |
| IPQCRLTPL | B*07 | 22 |
| YNLRRGTAL | B*08 | 23 |
| HPVGEADYFEY | B*35 | 24 |
| LSRLPFGMA | B*57 | 25 |
| FVYGGSKTSL | Cw*03 | 26 |

In some embodiments, the peptides provided herein comprise two or more of the EBV epitopes. In some embodiments, the peptides provided herein comprise at least 2, 3, 4, 5, 6, 7, 8, 9, 10, 11, 12, 13, 14, 15, 16, 17, 18, 19 or 20 EBV epitopes. For example, in some embodiments, the peptide provided herein comprises two or more of the EBV epitopes connected by linkers (e.g., polypeptide linkers).

In some embodiments, the sequence of the peptides comprises an EBV viral protein sequence except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a TCR and a peptide containing the amino acid sequence presented on an MHC. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

In some embodiments, the peptides provided herein comprise a sequence that is at least 80%, 85%, 90%, 95% or 100% identical to an EBV viral protein sequence (e.g., the sequence of a fragment of an EBV viral protein). To determine the percent identity of two amino acid sequences, the sequences are aligned for optimal comparison purposes (e.g., gaps can be introduced in one or both of a first and a second amino acid sequence for optimal alignment and non-identical sequences can be disregarded for comparison purposes). The amino acid residues at corresponding amino acid positions are then compared. When a position in the first sequence is occupied by the same amino acid residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

In some embodiments, the peptide is chimeric or fusion peptide. As used herein, a “chimeric peptide” or “fusion peptide” comprises a peptide having a sequence provided herein linked to a distinct peptide having sequence to which it is not linked in nature. For example, the distinct peptide can be fused to the N-terminus or C-terminus of the peptide provided herein either directly, through a peptide bond, or indirectly through a chemical linker. In some embodiments, the peptide of the provided herein is linked to another peptide comprising a distinct EBV epitopes. In some embodiments, the peptide provided herein is linked to peptides comprising epitopes from other viral and/or infectious diseases.

A chimeric or fusion peptide provided herein can be produced by standard recombinant DNA techniques. For example, DNA fragments coding for the different peptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The peptides provided herein can be isolated from cells or tissue sources by an appropriate purification scheme using standard protein purification techniques, and can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

In certain aspects, provided herein are nucleic acid molecules encoding the peptides described herein. In some embodiments, the nucleic acid molecule is a vector. In some embodiments, the nucleic acid molecule is a viral vector, such as an adenovirus based expression vector, that comprises the nucleic acid molecules described herein. In some embodiments, the vector provided herein encodes a plurality of epitopes provided herein (e.g., as a polyepitope). In some embodiments, the vector provided herein encodes at least 2, 3, 4, 5, 6, 7, 8, 9, 10, 11, 12, 13, 14, 15, 16, 17, 18, 19 or 20 epitopes provided herein (e.g., epitopes provided in Table 1).

In some embodiments, the vector is a viral vector (e.g. an adenovirus, such as AdE1-LMPpoly). The AdE1-LMPpoly vector encodes a polyepitope of defined CTL epitopes from LMP1 and LMP2 fused to a Gly-Ala repeat-depleted EBNA1 sequence. The AdE1-LMPpoly vector is described, for example, in Smith et al., Cancer Research 72:1116 (2012); Duraiswamy et al., Cancer Research 64:1483-9 (2004); and Smith et al., J. Immunol 117:4897-4906 (2006), each of which is hereby incorporated by reference.

As used herein, the term “vector,” refers to a nucleic acid molecule capable of transporting another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operably linked to one or more regulatory sequences (e.g., a promoter) in an expression vector. In some embodiments, the cell transcribes the nucleic acid provided herein and thereby expresses a peptide described herein. The nucleic acid molecule can be integrated into the genome of the cell or it can be extrachromasomal.

In some embodiments, provided herein are cells that contain a nucleic acid described herein (e.g., a nucleic acid encoding a peptide described herein). The cell can be, for example, prokaryotic, eukaryotic, mammalian, avian, murine and/or human. In some embodiments, the cell is a mammalian cell. In some embodiments the cell is an APC (e.g. an antigen-presenting T cell, a dendritic cell, a B cell, or an aK562 cell). In the present methods, a nucleic acid described herein can be administered to the cell, for example, as nucleic acid without delivery vehicle, in combination with a delivery reagent. In some embodiments, any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. In some embodiments of the methods described herein, liposomes are used to deliver a nucleic acid to a cell or subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

### Cytotoxic T Cells

Provided herein are methods of treating autoimmune diseases (e.g., MS, SAD, IBD) by administering to the subject allogeneic or autologous CTLs expressing a T cell receptor that specifically binds to an EBV peptide presented on a class I MHC. In some embodiments, the CTLs are from a cell bank. In some embodiments, the MHC is a class I MHC. In some embodiment, the class II MHC has an α chain polypeptide that is HLA-DMA, HLA-DOA, HLA-DPA, HLA-DQA or HLA-DRA. In some embodiments, the class II MHC has a β chain polypeptide that is HLA-DMB, HLA-DOB, HLA-DPB, HLA-DQB or HLA-DRB. In some embodiments, the CTLs are stored in a cell library or bank before they are administered to the subject.

In some embodiments, provided herein are APCs that present a peptide described herein (e.g., a peptide comprising a LMP1, LMP2A, or EBNA1 epitope sequence). In some embodiments the APCs are B cells, antigen presenting T-cells, dendritic cells, or artificial antigen-presenting cells (e.g., aK562 cells).

Dendritic cells for use in the process may be prepared by taking PBMCs from a patient sample and adhering them to plastic. Generally, the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then transduced with one or more of the peptides provided herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the one or more of the EBV epitopes described herein comprising contacting an APC with a peptide comprising a EBV epitope and/or with a nucleic acid encoding a EBV epitope. In some embodiments, the APCs are irradiated. In some embodiments, the APCs that present a peptide described herein (e.g., a peptide comprising a LMP1, LMP2A, or EBNA1 epitope sequence). A cell presenting a peptide described herein can be produced by standard techniques known in the art. For example, a cell may be pulsed to encourage peptide uptake. In some embodiments, the cells are transfected with a nucleic acid encoding a peptide provided herein. Provided herein are methods of producing antigen-presenting cells (APCs), comprising pulsing a cell with the peptides described herein. Exemplary examples of producing antigen presenting cells can be found in WO2013088114, hereby incorporated in its entirety.

In some embodiments, provided herein are T cells (e.g., CD4 T cells and/or CD8 T cells) that express a TCR (e.g., an αβ TCR or a γδ TCR) that recognizes a peptide described herein presented on a MHC. In some embodiments, the T cell is a CD8 T cell (a CTL) that expresses a TCR that recognizes a peptide described herein presented on a class I MHC. In some embodiments, the T cell is a CD4 T cell (a helper T cell) that recognizes a peptide described herein presented on a class II MHC.

In some embodiments, provided herein are methods of generating, activating and/or inducing proliferation of T cells (e.g., CTLs) that recognize one or more of the EBV epitopes described herein. In some embodiments, a sample comprising CTLs (i.e., a PBMC sample) is incubated in culture with an APC provided herein (e.g., an APC that presents a peptide comprising an EBV epitope on a class I MHC complex, such as the virally transduced PBMCs described herein). In some embodiments, the APCs are autologous to the subject from whom the T cells were obtained. In some embodiments, the APCs are not autologous to the subject from whom the T cells were obtained. In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some embodiments, provided herein are compositions (e.g., therapeutic compositions) comprising T cells and/or APCs provided herein used to treat and/or prevent an autoimmune disease in a subject by administering to the subject an effective amount of the composition. In some aspects, provided herein are methods of treating autoimmune disorders using a composition (e.g., a pharmaceutical composition, such compositions comprising allogeneic CTLs). In some embodiments, the composition includes a combination of multiple (e.g., two or more) CTLs provided herein.

### Therapeutic Methods

In some embodiments, the provided herein are methods of treating an autoimmune disorder in a subject by administering to the subject allogeneic CTLs provided herein. In some embodiments, the allogenic CTLs are selected from a cell bank (e.g., a pre-generated third party donor derived bank of epitope-specific CTLs).

In some embodiments, the methods provided herein can be used to treat any autoimmune disease. Examples of autoimmune diseases include, for example, glomerular nephritis, arthritis, dilated cardiomyopathy-like disease, ulcerous colitis, Sjogren syndrome, Crohn disease, systemic erythematodes, chronic rheumatoid arthritis, juvenile rheumatoid arthritis, Still's disease, multiple sclerosis, psoriasis, allergic contact dermatitis, polymyositis, pachyderma, periarteritis nodosa, rheumatic fever, vitiligo vulgaris, Behcet disease, Hashimoto disease, Addison disease, dermatomyositis, myasthenia gravis, Reiter syndrome, Graves' disease, anaemia perniciosa, sterility disease, pemphigus, autoimmune thrombopenic purpura, autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus erythematosus, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, lymphopenia, mixed connective tissue disease, pemphigoid, pemphigus vulgaris, pernicious anemia, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, type I diabetes, ulcerative colitis and Wegener's granulomatosis.

In some embodiments, the methods provided herein are used to treat MS. In some embodiments, the MS is relapsing-remitting MS, secondary progressive MS, primary progressive MS or progressively relapsing MS.

In some embodiments, the methods provided herein are used to treat a SAD. For example, in certain embodiments, the methods provided herein are used to treat rheumatoid arthritis, systemic lupus erythematosus and/or Sjögren's syndrome.

In some embodiments, the methods provided herein are used to treat IBD. For example, in certain embodiments the methods provided herein are used to treat Crohn's disease (regional bowel disease, e.g., inactive and active forms), celiac disease (e.g., inactive or active forms) and/or ulcerative colitis (e.g., inactive and active forms). In some embodiments, the methods provided herein are used to treat irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and/or primary sclerosing cholangitis.

In some embodiments, provided herein are methods of treating a cancer in a subject by administering to the subject a therapeutic CTL preparation as described herein.

In some embodiments, the methods provided herein can be used to treat any cancer. For example, in some embodiments, the methods and CTLs described herein may be used to treat any cancerous or pre-cancerous tumor. In some embodiments, the cancer includes a solid tumor. In some embodiments, cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods provided herein are used to treat EBV-associated cancer. In some embodiments, the EBV-associated cancer is EBV-associated NPC. In some embodiments, the EBV associated cancer is post-transplant lymphoproliferative disorder (PTLD), NK/T cell lymphoma, EBV+ gastric cancer, or EBV+ leiomyosarcoma.

In some embodiments, the subject has been exposed to a virus (e.g., EBV) such that virus particles are detectable in the subject's blood. In some embodiments, the method further comprises measuring viral load in the subject (e.g., before or after administering the peptide specific CTLs to the subject). Determining viral load in a subject may be a good prognostic marker for immunotherapy effectiveness. In some embodiments, selecting CTLs further comprises determining the number of viral DNA copies in the subject (e.g., in a tissue or blood sample). In some embodiments, viral load is measured two or more times.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some embodiments, the method includes selecting allogeneic CTLs from a cell bank (e.g., a pre-generated third party donor derived bank of epitope specific CTLs). In some embodiments, the CTLs are selected because they express a TCR restricted to a class I MHC that is encoded by an HLA allele that is present in the subject. In some embodiments, the CTLs are selected if the CTLs and subject share at least 2 (e.g., at least 3, at least 4, at least 5, at least 6) HLA alleles and the CTLs are restricted through a shared HLA allele. In some embodiments, the method comprises testing the TCR repertoire of the pre-generated third-party-donor-derived epitope-specific T cells (i.e., allogeneic T cells) with flow cytometry. In some embodiments epitope-specific T cells are detected using a tetramer assay, an ELISA assay, a western blot assay, a fluorescent microscopy assay, an Edman degradation assay and/or a mass spectrometry assay (e.g., protein sequencing). In some embodiments, the TCR repertoire is analyzed using a nucleic acid probe, a nucleic acid amplification assay and/or a sequencing assay.

### EXAMPLES

### Example 1: Generating Epitope Specific CTLs and Viral Replication Assay

Allogeneic latency-2 EBV-targeted cytotoxic T lymphocytes (allogeneic L2 EBV CTLs), or ATA188 (allogeneic EBV-CTL product), are HLA-matched, in vitro-expanded, antigen-specific T cells specific for EBV protein antigens including latent membrane protein 1 (LMP1), LMP2, and EBNA1. ATA188 is produced from the peripheral blood mononuclear cells (PBMCs) of healthy EBV seropositive donors.

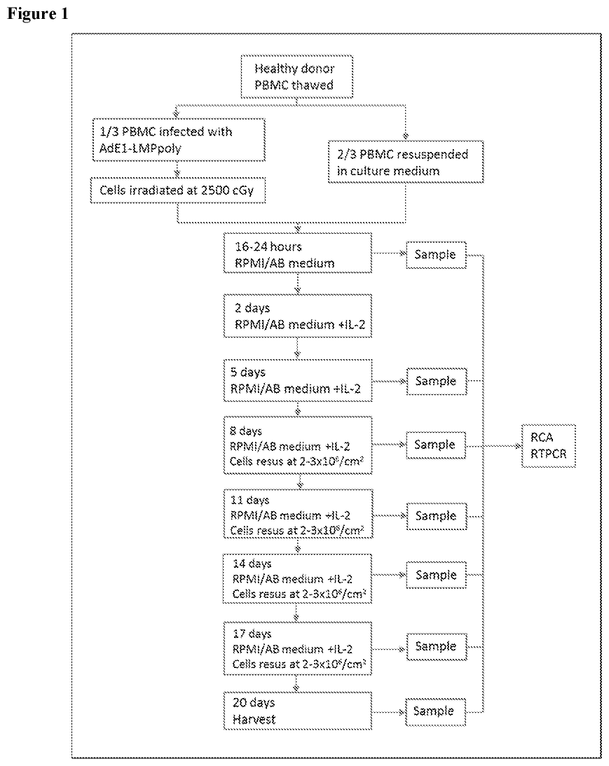

Frozen PBMC from healthy donors were thawed and recovered into RPMI medium. Cells were split into two fractions; ⅓ of cells (Stimulators) were infected with AdE1-LMPpoly adenovirus for one hour at 37° C. Stimulators were then washed twice and resuspended in RPMI/AB serum culture medium and irradiated at 2500 cGy (2500 rads). The remaining ⅔ of cells (Responders) were transferred to RPMI/AB serum medium and held at 37° C. until they could be mixed with the Stimulators. Cultures were initiated in 6 well (10 cm²) GRex culture plates at a ratio of 9×10⁶ Stimulator cells to 2.1×10⁷ Responder cells. One well was removed and the contents sampled on each of days 1, 5, 8, 11, 14, 17 and 20. The samples for testing were split between the viral replication and RT-PCR assays. It is expected that active virus will be present at early stages of the manufacturing process as this leads to presentation of viral epitopes to T cells (responder cells) by antigen presenting cells (stimulator cells), and should decrease as the non-proliferating stimulator cells eventually die. However, the aforementioned assays were used to determine if active virus (e.g., AdE1-LMPpoly) and/or viral mRNA could still be detected at later time points of the CTL culture and/or in the final CTL product. A summary of the experimental outline is shown in FIG. 1.

### Example 2: AdE1-LMPpoly and Viral Replication Assay

Two reporter cell lines are used to detect residual virus, AD293 and A549. The AD293 cell line (available from Cell Biolabs, Inc., Catalog #AD-100) is a permanent line established from primary embryonic human kidney transformed with human adenovirus type 5 DNA. These cells express the genes encoded by the E1 region of adenovirus (Ela and E1b) allowing these cells to complement the E1-deletion in recombinant adenoviral vectors, allowing viral replication of replication incompetent AdE1-LMP poly. The A549 line (available from Sigma-Aldrich©) is a permanent line derived from explanted cultures of human lung cancer tissue suitable as a negative control in assays to measure the replication of adenoviruses that lack E1A (e.g., cell line AD293) and as a target cell line to detect replication competent adenoviruses. A549 cells have been well characterized through their use in a wide variety of molecular studies, such as anti-tumor drug permeability and efficacy analysis, infection assays, respiratory immunotoxicity tests, cell senescence studies, and cytokine expression profiling. Cytopathic effects (e.g., plaque formation) are assessed on each cell line along with control AdE1-LMPpoly and wild type adenovirus to determine if replication competent and/or incompetent adenovirus are present in CTL cultures and/or preparations.

Pre-Assay Preparation:

Cell culture medium comprising Dulbecco's modified Eagle medium (DMEM), 10% foetal calf serum (FCS), and gentamicin was made fresh, the day of use. Vials of each cell line (AD293 and A549) were retrieved from liquid nitrogen storage and immediately held in dry ice prior to thawing. The vials were quickly thawed in a 37° C. water bath and were each immediately pipetted into 9 ml of cell culture medium to be centrifuged at 400 g for 10 minutes. The supernatant was aspirated and each cell line was resuspended in 10 ml of cell culture medium. Each of the AD293 and A549 cells were pipetted into labelled T25 flasks and incubated for up to 3 days (37° C./6.5%±1% CO₂).

Sample Preparation:

Samples from initiated cultures (e.g., 1×10⁶ cells for autologous CTL expansion cultures and 1×10⁷ cells for allogeneic CTL expansion cultures) were centrifuged at 400 g for 10 minutes. The supernatant was aspirated and the cell pellet resuspended in 1 mL of serum-free (i.e., no FCS) DMEM.

A bath comprising ethanol and dry ice was prepared. The resuspended cell pellets were subjected to three cycles of freeze-thawing by alternatively placing the tube containing the cells in the ethanol-dry ice bath to freeze followed by thawing in a 37° C. water bath. The resulting lysate was centrifuged at 400 g for 10 minutes and the supernatant (cell lysate free of debris) was transferred to a new 10 mL tube. A further 2 mL of serum-free DMEM was added to achieve a total volume of 3 mL. This provided sufficient lysate for 10 test wells of a 96-well plate for each cell line (i.e., AD293 and A549) and a retention sample of approximately 1 mL for storage.

Assay Preparation:

After up to 3 days, cultures of AD293 and A549 cells that formed a smooth monolayer and were not over-confluent were removed from the incubator and moved to a biological safety cabinet to be passaged (split). Without disturbing the monolayer, culture flasks were washed with PBS and the monolayer dissociated with 3 mLs of versene for approximately 10 minutes. Once dislodged, cells were transferred to a tube with 12 mL of cell culture medium and vortexed to ensure resuspension. For cell counting, 50p of the cell suspension was diluted in 50 μL of trypan blue, from which 10 μL was transferred to a haemocytometer. Based on the cell counts, AD293 cells and A549 cells were diluted to achieve a cell concentration of 0.5×10⁴ cells and 0.2×10⁴ cells per 100 μL culture medium, respectively.

To each well of a flat bottom 96 well plate, 100 μL of the AD293 cell culture (0.5×10⁴ cells) were added. Similarly, in another plate, 100 uL of the A549 cell culture (0.2×10⁴ cells) were added to each well. The plates were then wrapped in parafilm to prevent evaporation and incubated overnight at 37° C./6.5%±1% CO₂. The following day, plates were examined for smooth monolayers with 50-80% cofluency. Plates with greater than 80% confluency were discarded and new plates prepared.

Assay

The following day, plates were examined for smooth monolayers with 50-80% confluency. Plates with greater than 80% confluency were discarded and new plates prepared. To each of 10 wells of each plate (i.e., AD293 and A549) 100 μL of the sample lysate was added (i.e., test wells). Three 10-well sets of control wells (i.e., one negative and two positive controls) were also prepared in each plate as follows. Negative control wells received only 100 μL of serum-free DMEM and no sample lysate. Positive viral control wells were prepared in wells not adjacent to the test wells as follows. A suspension of AdE1-LMPpoly adenoviral particles was prepared from AdE1-LMPpoly stock VMC606 (Working virus bank) by making a 1/1000 dilution, i.e., 10 μL of AdE1-LMPpoly was diluted in 9.990 mL of serum-free DMEM and mixed thoroughly. Ten wells from each plate received 100 μL of the diluted AdE1-LMPpoly suspension as a positive control for the growth of AdE1-LMPpoly on the AD293 cells. A suspension of Human Adenovirus 5 (ATCC® VR-1516™) adenoviral particles was prepared by pipetting 10 μL of Human Adenovirus 5 stock into 9.99 mL of serum-free DMEM and mixing well. A second dilution was prepared by taking 10 μL from the first dilution and adding it to 9.99 mL of serum-free DMEM, thoroughly mixing the suspension. A further 10 wells on each 96-well received 100 μL of the diluted Human Adenovirus 5 suspension as a positive control for replication competent adenovirus. The 96-well plates were wrapped in parafilm and returned to the incubator (37° C./6.5%±1% CO₂) for 10 to 12 days.

Using microscopy, each well was assessed for cytopathic effects (CPE), e.g., rounding of cells and/or clearing of the monolayer (e.g., plaque formation). For each set of replicate wells for each cell line, the number of wells positive for CPE were counted, and the percentage of positive wells calculated. Briefly, for the assay to pass there should be no positive wells in the uninfected cell viability control wells in either AD293 or A549 cells (negative control). Test wells were scored as positive for CPE where the rounding of cells and/or clearing of the monolayer (e.g., plaque formation) were visibly different from the control wells. Positive control wells for AdE1-LMPpoly should be scored 100% positive in AD293 cultures. There should be no positive wells for the AdE1-LMPpoly in A549 wells as this virus is non-competent and will not grow in these cells. Positive control wells for Human Adenovirus 5 should be 100% positive in both AD293 and A549 cells as this virus is replication competent and will grow on both cell lines. For the test sample to have passed there should be no positive wells in the sample lysate wells on the A549 cells as this would indicate contamination with competent (infectious) adenovirus.

Results

The AdE1-LMPpoly was detected in the permissive AD293 cell line by cytopathic effects on day 1 of all three T cell culture time courses. Only one of the three cultures (AT0019) had detectable AdE1-LMPpoly on days 5 and 8. None of the cultures had detectable AdE1-LMPpoly after day 8.

Detection of replication competent adenovirus in the A549 cell line was not possible at any time point from the T cell cultures (Table 2).

Positive controls for both AdE1-LMPpoly and replication competent adenovirus (strain AD-5) were included in the experiment (Table 3).

TABLE 2
Results of detection of replication competent adenovirus
and replication incompetent AdE1-LMPpoly in time course
post infection. Ten wells were cultured and scored
for cytopathic effect for each test condition.

| | | Detection of Replication | Detection of AdE1- |
|---|---|---|---|
| Time | Donor | Competent Adenovirus | LMPpoly (AD293 |
| Point | Designation | (A549 cytopathic effect) | cytopathic effect) |
| Day 1 | AT0019 | 0 | 10 (100%) |
| | AT0028 | 0 | 10 (100%) |
| | AT0035 | 0 | 10 (100%) |
| Day 5 | AT0019 | 0 | 4 (40%) |
| | AT0028 | 0 | 0 |
| | AT0035 | N/D | N/D |
| Day 8 | AT0019 | 0 | 1 (10%) |
| | AT0028 | 0 | 0 |
| | AT0035 | 0 | 0 |
| Day 11 | AT0019 | 0 | 0 |
| | AT0028 | 0 | 0 |
| | AT0035 | 0 | 0 |
| Day 14 | AT0019 | 0 | 0 |
| | AT0028 | 0 | 0 |
| | AT0035 | 0 | 0 |
| Day 17 | AT0019 | 0 | 0 |
| | AT0028 | 0 | 0 |
| | AT0035 | 0 | 0 |
| Day 20 | AT0019 | 0 | 0 |
| | AT0028 | 0 | 0 |
| | AT0035 | 0 | 0 |

N/D denotes no data collected at this time point.

TABLE 3
Results from controls in the detection of replication
competent adenovirus. The AD-5 strain of adenovirus
was used as the replication competent adenovirus positive
control. Results are based on scoring of cytopathic
effects on 10 wells with cell lines as indicated.

| | A549 (cytopathic | AD293 (cytophathic |
|---|---|---|
| Sample | effect) | effect) |
| Medium alone | 0 | 0 |
| (negative control) | | |
| AdE1-LMPpoly | 0 | 10 (100%) |
| (positive control) | | |
| AD-5 | 10 (100%) | 10 (100%) |
| (positive control) | | |

Cellular RNA was extracted using a Qiagen RNeasy kit (cat #74136) and RNA quantity determined using a Nanodrop spectrophotometer. RNA was DNase treated to remove residual DNA prior to PCR. Equivalent RNA amounts were used for each PCR (30 ng). A Qiagen OneStep RT-PCR kit was used for the PCR reactions. The one step RT-PCR reaction has a 30 minute reverse transcriptase utilizing a specific 3′ primer. A quantitative PCR reaction was run for 40 cycles.

Primers and Probes

Fluorescent 6-carboxyfluorescein (FAM) and black hole quencher 1 (BQ1) fluorescent labeled oligo probes and primers are shown in Table 4. The E1PT primer set was designed to PCR across the junction of the EBNA1 and polyepitope region of the E1-LMPpoly gene. It is designed to discriminate between any wild type endogenous adenovirus and the AdE1-LMPpoly. A commercial primer set was purchased from ThermoFisher (Cat #4333764F) for the control GAPD housekeeping gene.

TABLE 4
Primer sets used for RT-PCR. 6FAM and BHQ1
are the fluorescent probes for
Taqman analysis.

| Gene/primer | Primer Sequence | Sequence ID |
|---|---|---|
| E1PT Forward | GGTGATGGAGATGAGGGTGA | SEQ ID NO: 27 |
| E1PT Reverse | TCCTCAGGCACATGAACACG | SEQ ID NO: 28 |
| E1PT Probe | [6FAM]GGAGGAATTCCCC | SEQ ID NO: 29 |
| | TACCTGT[BHQ1] | |

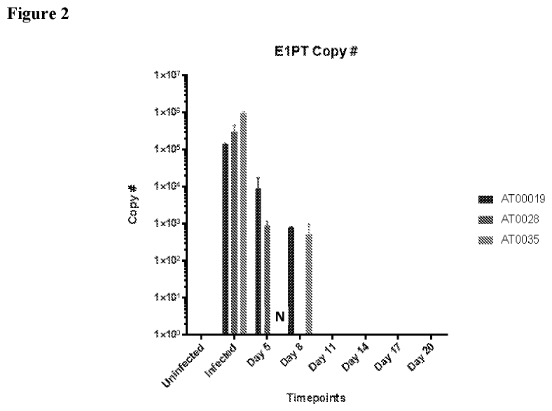

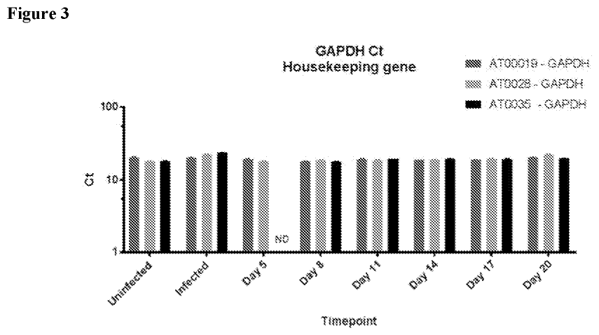

AdE1-LMPpoly specific cDNA (mRNA) could be detected in PBMC between 24 hours and 8 days of culture (FIG. 2). The copy number reduced during this period. Signal was undetectable after day 8 (below detection limit of 50 copies). The cellular GAPDH gene was used as an internal loading control to demonstrate comparable quantities of mRNA at every time point. mRNA loading was shown to be comparable at all time points and results are shown in FIG. 3.

Results

E1LMPpoly mRNA transcripts could be detected from as early as 24 hours up until day 8 of culture. The expression of AdE1-LMPpoly genes by infected Stimulator cells is required for the antigen presenting function of Stimulator Cells and thus expression of these genes is expected for a period of time after culture initiation.

Residual AdE1-LMPpoly virus could be detected in the permissive AD293 cell line from culture supernatants up to day 8 which is in agreement with the RT-PCR result. No replication competent adenovirus from the T cell cultures could be detected at any time point (i.e. there had been no recombination events leading to the reversion of the replication competent adenovirus to competent adenovirus).

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of identifying a preparation of responder cells comprising cytotoxic T lymphocytes (CTLs) as essentially free of an active virus, comprising:
i) preparing a culture of responder cells comprising virally transduced stimulator cells;
ii) collecting at least one sample from said culture;
iii) assessing the ability of said sample to form viral plaques on a susceptible reporter cell line; and
iv) determining the presence or absence of a viral mRNA in said sample,
wherein if the samples do not form viral plaques and said viral mRNA is not detected, the culture is identified as being essentially free of the active virus.

2. The method of claim 1, further comprising incubating the stimulator cells with one or more cytokines prior to culturing with responder cells.

3. The method of claim 1, further comprising irradiating the stimulator cells with gamma rays prior to culturing with responder cells.

4. The method of claim 1, wherein the responder cells and the stimulator cells each comprise peripheral blood mononuclear cells (PBMC).

5. The method of claim 1, wherein the responder cells and the stimulator cells are each derived from the same donor or are each derived from a different donor.

6. (canceled)

7. The method of claim 1, wherein the stimulator cells comprise B cells, antigen-presenting T-cells, dendritic cells, artificial antigen-presenting cells, and/or aK562 cells.

8. The method of claim 1, wherein the stimulator cells express a replication incompetent adenoviral vector comprising a nucleic acid sequence encoding one or more herpesvirus antigens.

9-11. (canceled)

12. The method of claim 8, wherein the adenoviral vector is AdE1-LMPpoly.

13. (canceled)

14. The method of claim 1, comprising maintaining the responder cells in said culture for at least 24 hours, at least 5 days, at least 8 days, at least 11 days, at least 14 days, at least 17 days, or at least 20 days prior to performing the assessing and determining steps.

15-16. (canceled)

17. The method claim 14, further comprising harvesting the CTLs for processing as required prior to use and/or storage if the samples do not form viral plaques and said viral mRNA is not detected.

18. The method of claim 14, further comprising reseeding, excluding, or discarding the culture according to a predetermined protocol if the samples do form viral plaques and/or said viral mRNA is detected.

19. The method of claim 1, comprising identifying the preparation as essentially free of a virus by confirming that the sample does not form viral plaques in a reporter cell line susceptible to said virus, and optionally wherein the reporter cell line expresses endogenous E1 protein.

20. The method of claim 19, wherein the reporter cell line is A549 or AD293.

21. The method of claim 1, comprising identifying the preparation as essentially free of a recombinant, replication-incompetent virus by confirming that the sample does not form viral plaques in a reporter cell line susceptible to said virus, wherein the reporter cell line expresses endogenous E1 protein.

22-25. (canceled)

26. The method of claim 14, wherein the viral mRNA is detected using an RT-PCR assay comprising:
i) extracting RNA from the sample,
ii) preparing cDNA from the RNA extracted from step (i),
iii) amplifying the cDNA with a primer set unique and specific to the virus;
iv) hybridizing the amplified product of step (iii) with labeled probes; and
v) detecting and quantitating the hybridization of the labeled probes to the amplified product.

27. The method of claim 26, wherein the primer set is designed to hybridize to and amplify a nucleic acid sequence specific to AdE1-LMPpoly or a nucleic acid sequence common to wild-type endogenous virus.

28. (canceled)

29. The method of claim 27, wherein the primer set is SEQ ID NO: 27 and SEQ ID NO: 28.

30. The method claim 26, wherein the labeled probes are fluorescently labeled oligo probes comprising the sequence set forth in SEQ ID NO: 29.

31-33. (canceled)

34. The method of claim 17, wherein the preparation is essentially free from the active virus at CTL harvest.

35-65. (canceled)

66. A method of identifying a therapeutic preparation of cytotoxic T lymphocytes (CTLs) as suitable for administering to a recipient, comprising:
i) obtaining a sample of a therapeutic preparation of CTLs;
ii) assessing the ability of the preparation to form viral plaques on a plurality of reporter cell lines; and
iii) determining the presence or absence of a viral mRNA in the preparation, wherein the preparation is identified as suitable for administering to the recipient if the sample does not form viral plaques and said viral mRNA is not detected.

67. (canceled)

68. The method of claim 66, wherein the formation of viral plaques and/or presence of viral mRNA is due to an adenovirus.

69. The method of claim 68, wherein the adenovirus comprises a replication incompetent adenoviral vector comprising a nucleic acid sequence encoding one or more herpesvirus antigens.

70-71. (canceled)

72. The method of claim 68, wherein the adenovirus is AdE1-LMPpoly.

73. The method of claim 68, wherein the CTLs are derived from the recipient or from a donor that is not the recipient.

74-76. (canceled)

77. The method of claim 66, comprising identifying the preparation as essentially free of a virus by confirming that the sample does not form viral plaques in a reporter cell line susceptible to said virus, and optionally wherein the reporter cell line expresses endogenous E1 protein.

78. The method of claim 77, wherein the reporter cell line is A549 or AD293.

79. The method of claim 66, comprising identifying the preparation as essentially free of recombinant, replication-incompetent virus by confirming that the sample does not form viral plaques in a reporter cell line susceptible to said active virus, wherein the reporter cell line expresses endogenous E1 protein.

80-83. (canceled)

84. The method of claim 77, wherein the viral mRNA is detected using an RT-PCR assay comprising:
i) extracting RNA from the sample,
ii) preparing cDNA from the RNA extracted from step (i),
iii) amplifying the cDNA with a primer set unique and specific to the virus;
iv) hybridizing the amplified product of step (iii) with labeled probes; and
v) detecting and quantitating the hybridization of the labeled probes to the amplified product.

85. The method of claim 84, wherein the primer set is designed to hybridize to and amplify a nucleic acid sequence specific to AdE1-LMPpoly or a nucleic acid sequence common to a wild-type endogenous virus.

86. (canceled)

87. The method claim 85, wherein the primer set is SEQ ID NO: 27 and SEQ ID NO: 28.

88. The method of claim 87, wherein the labeled probes are fluorescently labeled oligo probes comprising the sequence set forth in SEQ ID NO: 29.

89-90. (canceled)

91. A method of treating an EBV antigen-associated condition in a human subject, comprising administering to the subject CTLs identified as being substantially free of active virus by the method of claim 66.

92. A method of treating an EBV antigen-associated condition in a human subject, comprising performing the method of claim 66 and administering CTLs identified as being substantially free of active virus to the subject.


SEQUENCE LISTING (29 sequences)

SEQ ID NO: 1 - protein, 376 aa, Human gammaherpesvirus 4
MDLDLERGPPGPRRPPRGPPLSSYIALALLLLLLALLFWLYIIMSNWTGGALLVLYAFALMLVIIILIIFIFRRDLLCPLGALCLLLLMITLLLIALWNLHGQALYLGIVLFIFGCLLVLGIWVYFLEILWRLGATIWQLLAFFLAFFLDILLLIIALYLQQNWWTLLVDLLWLLLFLAILIWMYYHGQRHSDEHHHDDSLPHPQQATDDSSNHSDSNSNEGRHHLLVSGAGDAPPLCSQNLGAPGGGPDNGPQDPDNTDDNGPQDPDNTDDNGPHDPLPQDPDNTDDNGPQDPDNTDDNGPHDPLPHNPSDSAGNDGGPPNLTEEVENKGGDRGPPSMTDGGGGDPHLPTLLLGTSGSGGDDDDPHGPVQLSYYD

SEQ ID NO: 2 - protein, 497 aa, Human gammaherpesvirus 4
MGSLEMVPMGAGPPSPGGDPDGDDGGNNSQYPSASGSDGNTPTPPNDEERESNEEPPPPYEDLDWGNGDRHSDYQPLGNQDPSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLTKILLARLFLYALALLLLASALIAGGSILQTNFKSLSSTEFIPNLFCMLLLIVAGILFILAILTEWGSGNRTYGPVFMCLGGLLTMVAGAVWLTVMTNTLLSAWILTAGFLIFLIGFALFGVIRCCRYCCYYCLTLESEERPPTPYRNTV

SEQ ID NO: 3 - protein, 238 aa, Human gammaherpesvirus 4
PFFHPVGEADYFEYLQEGGPDGEPDVPPGAIEQGPADDPGEGPSTGPRGQGDGGRRKKGGWFGKHRGQGGSNPKFENIAEGLRVLLARSHVERTTEEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 4 - protein, 9 aa, Human gammaherpesvirus 4
CLGGLLTMV

SEQ ID NO: 5 - protein, 9 aa, Human gammaherpesvirus 4
FLYALALLL

SEQ ID NO: 6 - protein, 9 aa, Human gammaherpesvirus 4
YLQQNWWTL

SEQ ID NO: 7 - protein, 9 aa, Human gammaherpesvirus 4
YLLEMLWRL

SEQ ID NO: 8 - protein, 9 aa, Human gammaherpesvirus 4
ALLVLYSFA

SEQ ID NO: 9 - protein, 9 aa, Human gammaherpesvirus 4
LLSAWILTA

SEQ ID NO: 10 - protein, 9 aa, Human gammaherpesvirus 4
LTAGFLIFL

SEQ ID NO: 11 - protein, 11 aa, Human gammaherpesvirus 4
SSCSSCPLSKI

SEQ ID NO: 12 - protein, 8 aa, Human gammaherpesvirus 4
PYLFWLAA

SEQ ID NO: 13 - protein, 9 aa, Human gammaherpesvirus 4
TYGPVFMCL

SEQ ID NO: 14 - protein, 10 aa, Human gammaherpesvirus 4
VMSNTLLSAW

SEQ ID NO: 15 - protein, 8 aa, Human gammaherpesvirus 4
CPLSKILL

SEQ ID NO: 16 - protein, 9 aa, Human gammaherpesvirus 4
RRRWRRLTV

SEQ ID NO: 17 - protein, 9 aa, Human gammaherpesvirus 4
IEDPPFNSL

SEQ ID NO: 18 - protein, 9 aa, Human gammaherpesvirus 4
IALYLQQNW

SEQ ID NO: 19 - protein, 9 aa, Human gammaherpesvirus 4
MSNTLLSAW

SEQ ID NO: 20 - protein, 9 aa, Human gammaherpesvirus 4
VLKDAIKDL

SEQ ID NO: 21 - protein, 9 aa, Human gammaherpesvirus 4
RPQKRPSCI

SEQ ID NO: 22 - protein, 9 aa, Human gammaherpesvirus 4
IPQCRLTPL

SEQ ID NO: 23 - protein, 9 aa, Human gammaherpesvirus 4
YNLRRGTAL

SEQ ID NO: 24 - protein, 11 aa, Human gammaherpesvirus 4
HPVGEADYFEY

SEQ ID NO: 25 - protein, 9 aa, Human gammaherpesvirus 4
LSRLPFGMA

SEQ ID NO: 26 - protein, 10 aa, Human gammaherpesvirus 4
FVYGGSKTSL

SEQ ID NO: 27 - DNA, 20 bp, Artificial Sequence
  Description of Artificial Sequence Synthetic primer
ggtgatggagatgagggtga

SEQ ID NO: 28 - DNA, 20 bp, Artificial Sequence
  Description of Artificial Sequence Synthetic primer
tcctcaggcacatgaacacg

SEQ ID NO: 29 - DNA, 20 bp, Artificial Sequence
  Description of Artificial Sequence Synthetic probe
ggaggaattcccctacctgt